(19) United States
(12) Patent Application Publication
(10) Pub. No.: US 2018/0028129 A1
(43) Pub. Date: Feb. 1, 2018

(54) Method, System, and Apparatus for Aligning Fixator Rings

(71) Applicants: Robert Ian Broadhead, Scarborough (GB); David Clive Broadhead, Auckland (NZ)

(72) Inventor: Robert Ian Broadhead, Scarborough (GB)

(73) Assignees: Robert Ian Broadhead, Scarborough (GB); David Clive Broadhead, Auckland (NZ)

(21) Appl. No.: 15/223,282

(22) Filed: Jul. 29, 2016

(51) Int. Cl. A61B 6/04; A61B 6/00; A61B 17/62

(52) U.S. Cl. CPC A61B 6/0492; A61B 17/62; A61B 6/547

(57) ABSTRACT

A preferred form of the invention relates to the field of orthopaedic medicine, and in particular to a means for quickly and easily achieving the desired position of the ring of an external fixator frame relative to the lens of an x-ray machine. A particularly preferred form of the invention relates to a device to assist radiographers in obtaining the desired alignment of external fixator frame rings in relation to the lens of an x-ray machine.

### FIELD OF INVENTION

A preferred form of the invention relates to the field of orthopaedic medicine, and in particular to a means for quickly and easily achieving the desired position of the ring of an external fixator frame relative to the lens of an x-ray machine. A particularly preferred form of the invention relates to a device to assist radiographers in obtaining the desired alignment of external fixator frame rings in relation to the lens of an x-ray machine.

### BACKGROUND

External fixator frames have been widely used to treat fractures and for other purposes for many years. They consist of two or more circular rings which are attached to the bone on either side of the treatment area and are connected to each other via connecting rods. The lengths of the rods can be adjusted, thereby adjusting the position of the two parts of the bone relative to each other. An example is shown at FIG. 23. Whilst there have been several advancements in the design of external fixator frames, the fundamental problem of achieving consistent alignment of the reference ring during x-ray imaging, so that progress can be assessed and the best course of future treatment determined, remains.

For the purpose of taking an x-ray image of a bone, one of the rings of the circular external fixator frame is nominated as the reference ring and a master tab may be attached to this ring as a specific point of reference. The normal procedure is for the patient to sit or lie on a bed. The medical staff then position the limb and reference ring to achieve the desired alignment and orientation of that ring to the lens of the x-ray machine. The aim is to take two x-rays. In both cases the desired position of the reference ring is orthogonal to the lens of the x-ray machine.

One x-ray is taken from directly above the master tab. A second x-ray is taken from 90 degrees away from the first. Generally speaking; a top view and a side view. The two views are normally achieved by leaving the patient in the same position and changing the position of the x-ray machine, from above the treatment area to the side of it, for example. The same result may also be achieved by leaving the x-ray machine in the same position and moving the patient.

The x-ray machine projects a beam and guideline “cross hair” onto the target area. This indicates the area which will be captured on the x-ray. The staff then ‘do their best’, normally by means of visual estimation, to achieve the desired orthogonal orientation of the reference ring to the x-ray machine before leaving the room to avoid exposure to x-rays.

The limb to which the external fixator frame is attached obscures the view of one portion of the reference ring so that only part of the ring is visible to the staff. It is normally not possible for the staff to see the ring on either side of the limb and it is therefore difficult to achieve the desired orthogonal alignment of the ring in relation to the lens of the x-ray machine. There is often no clear indicator as to whether correct alignment has been achieved, either during the first part of the process, when medical staff are positioning a patient, or the second when the medical staff have left the room, with the patient typically just being instructed not to move.

It is an object of a preferred embodiment of the current invention to go at least some way towards addressing the above problem. Whilst this object applies to the preferred embodiment, it should be understood that it is not intended to limit the scope of the claims. This is because the object of the invention per se is simply to provide the public with a useful choice.

The term comprising and derivatives thereof, e.g. “comprises”, if and when used herein in relation to a combination of features should not be taken as excluding the possibility that the combination may have further unspecified features. For example, a statement than an arrangement “comprises” certain parts does not mean that it cannot also, optionally, have additional parts.

### SUMMARY OF INVENTION

According to a first aspect of the invention there is a method of aligning an external fixator frame ring and a body part imaging device with respect to one another, comprising causing a light (eg a line of light) to be projected from the position of the ring to indicate the planar disposition of the ring, and using the light (eg the line of light) to align the ring and imaging device with respect to one another.

Optionally the body part imaging device comprises an x-ray imaging machine.

Optionally the line of light is emitted as a plane of light.

Optionally the line of light comprises laser light.

Optionally the line of light is in the same plane as the ring.

Optionally the line of light is projected by light emitting means secured to the ring.

Optionally the light emitting means projects two lines of light to create a cross-hair, the cross-hair indicating both the planar and rotational disposition of the ring.

Optionally the plane of light is projected by a light emitting means secured to the ring.

Optionally the light emitting means projects two lines of light; the first being in the same plane as the ring and the second being perpendicular to the first, creating a “cross-hair” which indicates both the planar and rotational disposition the ring.

According to a further aspect of the invention there is provided an external fixator frame comprising:
an external fixator frame ring; and
a laser alignment device having a laser line generating module and two fixing arms,
   the fixing arms enabling the device to be removably attached to the ring so that the device projects a laser line in the same plane as the ring.

According to another aspect of the invention there is provided an external fixator frame comprising:
a circular external frame ring; and
a laser alignment device having an attachment structure and a laser module housing;
   the laser module housing being removably attached to the attachment structure and the attachment structure being able to be removably attached to the ring in such a position that a laser line generating module housed by the laser module projects a laser cross-hair; one laser line of the cross-hair being in the same plane as the ring and a second one perpendicular to the first.

According to another aspect of the invention there is provided a circular external fixator frame comprising:
a ring; and
a laser module housing;
   the laser module housing being removably attached to the ring, the ring having locator holes in such a position that the laser module housing houses a laser line generating module which projects a laser cross-hair; one laser line of the cross-hair being in the same plane as the ring and a second one perpendicular to the first.

Optionally the housing is releasably secured to the ring by way of a knurled knob which draws the fixing arms together.

Optionally the laser line generating module produces an IEC60825 Class 2 laser line.

Optionally the laser line generating module uses batteries as its source of power.

Optionally the laser line generating module incorporates an “on-off” switch.

Optionally the housing is associated with an attachment structure that comprises a platform, arms, knuckles and fingers.

Optionally the arms are “handed” i.e. they are not the same.

Optionally there are two of the arms; a left arm and a right arm.

Optionally the tops of the arms are geared.

Optionally the tops of the left and right arms are geared together and pivoted within the platform, such that the perpendicular of the platform always bisects the angle made by the arms.

Optionally the arms are attached to the platform by pivot pins.

Optionally there are two of the pivot pins.

Optionally one of the pivot pins is large and the other is small.

Optionally the large pivot pin is used for the left arm.

Optionally the small pivot pin is used for the right arm.

Optionally the lower ends of the arms, which come into contact with the body, have rounded edges.

Optionally there are knuckles in the lower end of each arm.

Optionally there are two of these knuckles.

Optionally there are fingers extending from the knuckle of each arm.

Optionally there are two of these fingers extending from the knuckle of each arm.

Optionally the fingers are joined together by a tension spring.

Optionally the laser module housing incorporates registering pins.

Optionally there are two of the registering pins.

Optionally one of the registering pins is large and the other is small.

Optionally the platform incorporates locating holes.

Optionally there are two of the locating holes.

Optionally one of the locating holes is large and the other is small.

Optionally the large and small registering pins fit into the large and small locating holes respectively in the platform.

Optionally the laser line generating module produces an IEC60825 Class 2 laser cross-hair.

Optionally the laser line generating module uses batteries as its source of power.

Optionally the laser module housing incorporates an “on-off” switch.

According to a further aspect of the invention there is an alignment device having:
attachment means; and
light emitting means;
   the device being secured to an external fixator frame ring by way of the attachment means and activated so that the emitting means projects a line of light towards a body imaging machine.

Optionally the body imaging device comprises an x-ray imaging machine.

Optionally the line of light is indicative of the planar disposition of the ring.

Optionally the light is emitted as a plane of light.

Optionally the line of light comprises laser light.

Optionally the light emitting means also projects a second line of light towards the body imaging machine and wherein the second line is perpendicular to the first so that the two lines form a cross-hair.

Optionally the attachment means comprises a pair of arms engaged with the ring.

### BRIEF DESCRIPTION OF THE DRAWINGS

Some preferred forms of the invention will now be described by way of example and with reference to the accompanying drawings, of which:

FIG. 1 is a schematic side view showing a laser line alignment device;

FIG. 2 is a schematic plan view showing the laser line alignment device;

FIG. 3 is a schematic side view showing the laser line alignment device attached to a circular external fixator frame as it would be when below an x-ray imaging machine;

FIG. 4 is a schematic end view showing the laser line alignment device attached to the circular external fixator frame as it would be when below the x-ray imaging machine above;

FIG. 5 is a schematic inverted plan view showing a laser line from the device projected onto an x-ray machine projection head;

FIG. 6 is a schematic side view of a laser cross-hair alignment device attached to a circular external fixator frame;

FIG. 7 is a schematic end view of the laser cross-hair alignment device attached to the circular external fixator frame;

FIG. 8 is a schematic plan view of the laser cross-hair alignment device attached to the circular external fixator frame;

FIG. 9A is a schematic side view showing arms of the alignment device in two dispositions of use;

FIG. 9B is a schematic side view showing arms of the alignment device in a second disposition of use;

FIG. 10 is a schematic side view showing the arms when formed to be geared;

FIG. 11 is a schematic plan view showing the geared arms;

FIG. 12 is a schematic side view showing the laser alignment device;

FIG. 13 is a schematic side view showing a platform and pivot pins of the alignment device;

FIG. 14 is a schematic end view showing the platform and pivot pins;

FIG. 15 is a schematic plan view showing the platform and pivot pins;

FIG. 16 shows the components of a knuckle finger tension spring of the alignment device;

FIG. 17 is a schematic side view showing the knuckle finger tension spring in use;

FIG. 18 is a schematic end view showing the knuckle finger tension spring in use;

FIG. 19 is a schematic plan view showing the knuckle finger tension spring in use;

FIG. 20 is a schematic side view showing the external fixator frame with locator holes;

FIG. 21 is a schematic end view showing the external fixator frame with locator holes;

FIG. 22 is a schematic inverted plan view showing a laser cross-hair projected onto an x-ray machine projection head; and

FIG. 23 is an isometric view showing part of a fixator frame with one of its rings fitted to the bone of a patient's leg via pins.

### DETAILED DESCRIPTION

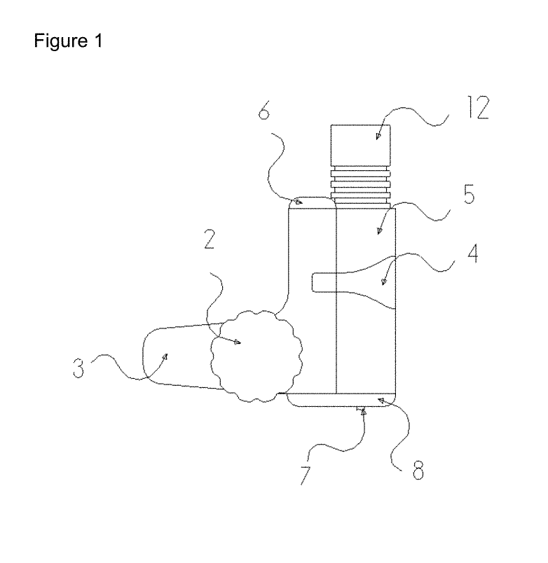

FIG. 1 shows the components of the laser line alignment device. A laser line is produced by a laser line generating module 12, which is contained inside a housing 5. The generating module 12 is held in the desired position by means of a clamp ring 4. The device incorporates batteries which are covered by the battery cap 6. It is switched on and off by means of an on-off switch 7 which is located within a switch cap 8. The device also incorporates two clamping arms 3.

When an external fixator frame is first fitted to the patient, one of the rings is nominated as the reference ring and it is preferable that this ring be orthogonal to an x-ray imaging machine each time an x-ray image is taken.

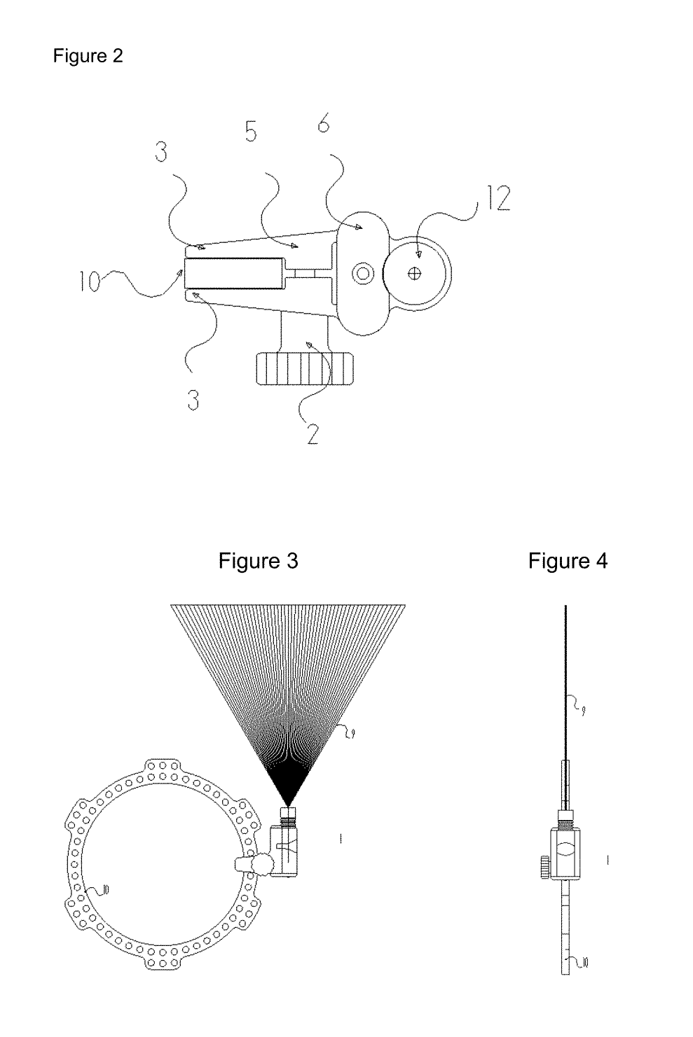

FIG. 2 shows how the laser line alignment device is attached to a circular external fixator frame reference ring 10 by means of a clamping knob 2 which draws together the two clamping arms 3 around said reference ring.

FIGS. 3 and 4 show two different views of the laser line alignment device 1 attached to the circular external fixator frame reference ring 10 and projecting a laser line of light 9 in the same plane as the reference ring.

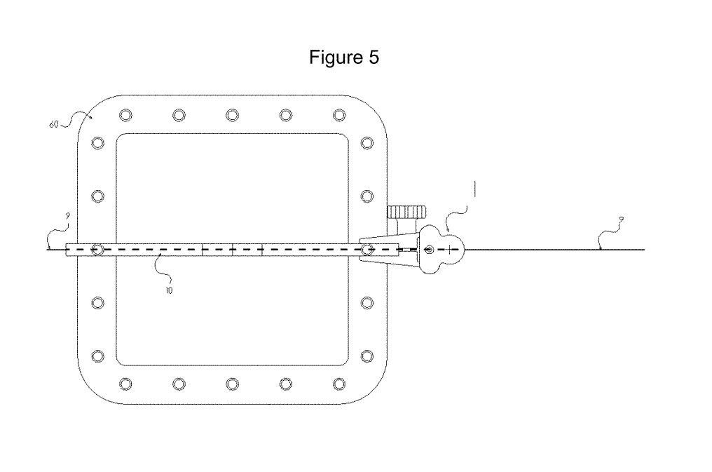

Without the use of the device it is difficult to achieve the desired orthogonal alignment of the ring 10 to the x-ray machine. FIG. 5 shows the laser line of light 9 being projected in the same plane as the ring and orthogonal to the cover of the x-ray machine 60. It is normal to take x-rays from two different angles; one from directly above and one from the side or 90 degrees removed from the first. The device can be positioned so that it will not obstruct the view of the treatment area. For example if the x-ray image is being taken from directly above the ring the device can be attached to the side, whereas if it is being taken from the side the device can be attached to the top. Once the desired alignment has been achieved, the patient can use the projected line as an ongoing guide to maintain alignment when the medical staff have left the room and until the x-ray image has been taken. Being quickly detachable also offers users the option of removing the device after the desired alignment has been achieved and before the x-ray is taken.

The laser line alignment device indicates the plane of the reference ring to which it is attached.

A laser cross-hair alignment device discussed below indicates the plane of the ring and the rotational position of the ring within that plane.

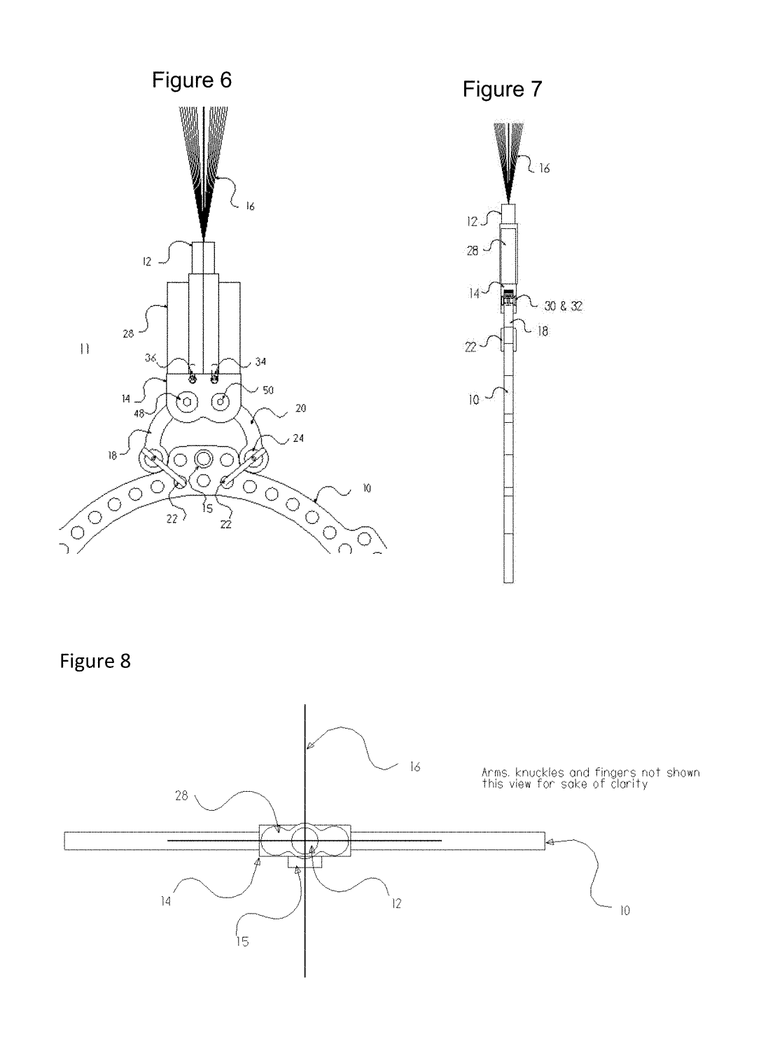

FIGS. 6, 7 and 8 show the laser cross-hair alignment device 11 mounted onto the reference ring 10 directly above a master tab 15. The master tab 15 is a reference point and is attached to the reference ring 10 and used by medical staff to achieve the desired position of the ring 10 for each x-ray image taken. This normally occurs with the master tab 15 in the uppermost position. Since the laser cross-hair alignment device is attached to the reference ring 10 directly above the master tab 15 and projects a cross-hair 16 comprising one line in the same plane as the reference ring and one perpendicular to it, and from directly above the master tab 15, it shows both the plane of the ring and the rotational position within that plane in relation to the x-ray device.

The components of the laser cross-hair alignment device will be described below.

External fixator frame rings normally have an inner ring of holes plus raised sections. The drawings show these raised sections as having two rows of three holes, one on top of the other.

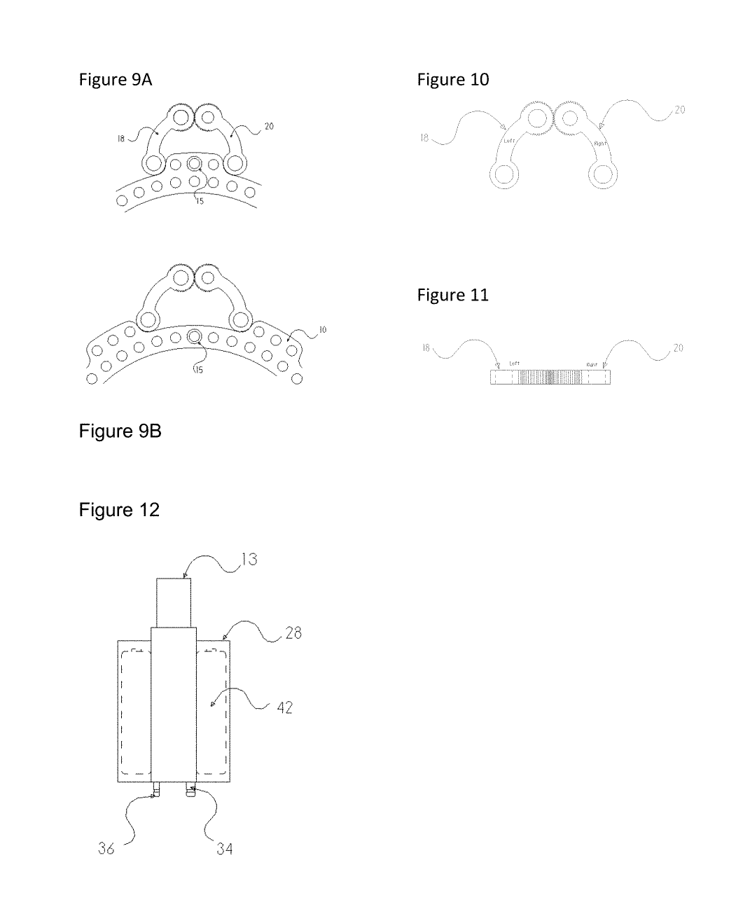

FIG. 9 shows the left arm 18 and right arm 20 of the cross-hair alignment device in two dispositions of use. In the upper drawing the master tab 15 is attached to the middle hole of the upper row of one of the raised sections of the reference ring 10. The left and right arms 18 and 20 are straddling either side of this raised section. In the lower drawing the master tab 15 is attached to the middle hole of the inner ring, between two raised sections of the reference ring 10. The bottom of the left and right arms 18 and 20 are abutting the edges of these two raised sections.

FIGS. 10 and 11 show the left arm 18 and the right arm 20. The two arms are geared together.

FIG. 12 shows a laser module housing for the cross-hair device 28. It comprises a laser cross-hair generating module 13 and two batteries 42, as well as a small registering pin 36 and a large registering pin 34.

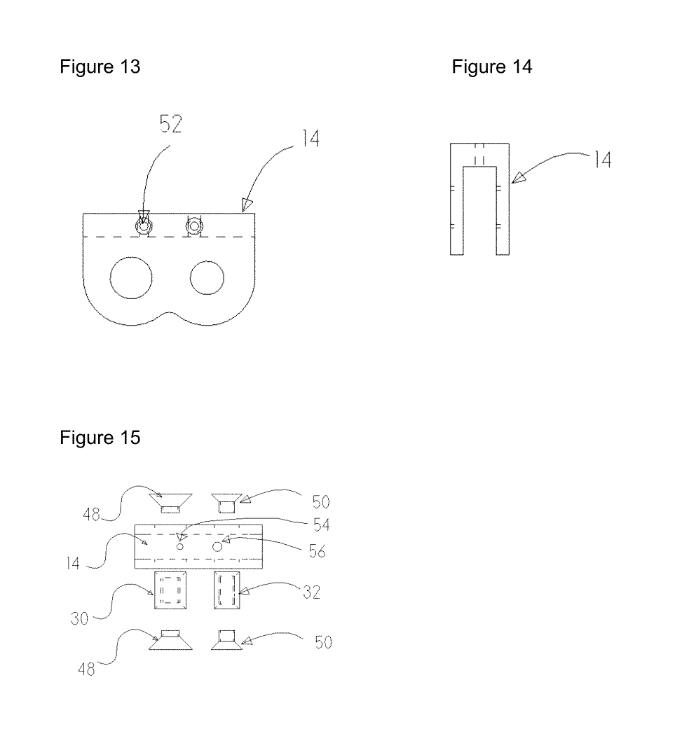

FIGS. 13, 14 and 15 show a platform 14, a large pivot pin 30, a small pivot pin 32, large pivot pin screws 48, small pivot pin screws 50, a small hole in the platform 54, a large hole in the platform 56 and sprung ball grub screws 52. The arms and pivot pins are “handed”, not interchangeable.

The left arm 18 is attached to the platform 14 by means of the large pivot pin 30 and large pivot pin screws 48. The right arm 20 is attached to the platform 14 by means of the small pivot pin 32 and small pivot pin screws 50. Similarly the holes in the platform are small and large and so are the registering pins in the bottom of the laser module housing for the cross hair device. The small registering pin 36 fits into the small hole 54 in the platform whilst the large registering pin 34 fits into the large hole 56. The sprung ball grub screws 52 secure the registering pins in place in the holes.

The tops of the left and right arms are geared together and pivoted within the platform, such that the perpendicular of the platform always bisects the angle made by the arms. The position of the laser cross hair generating module above the geared arms which are in turn above the master tab means that the projected cross hair displays the position of the master tab in relation to the x-ray machine in terms of both the plane and the rotational position within the plane.

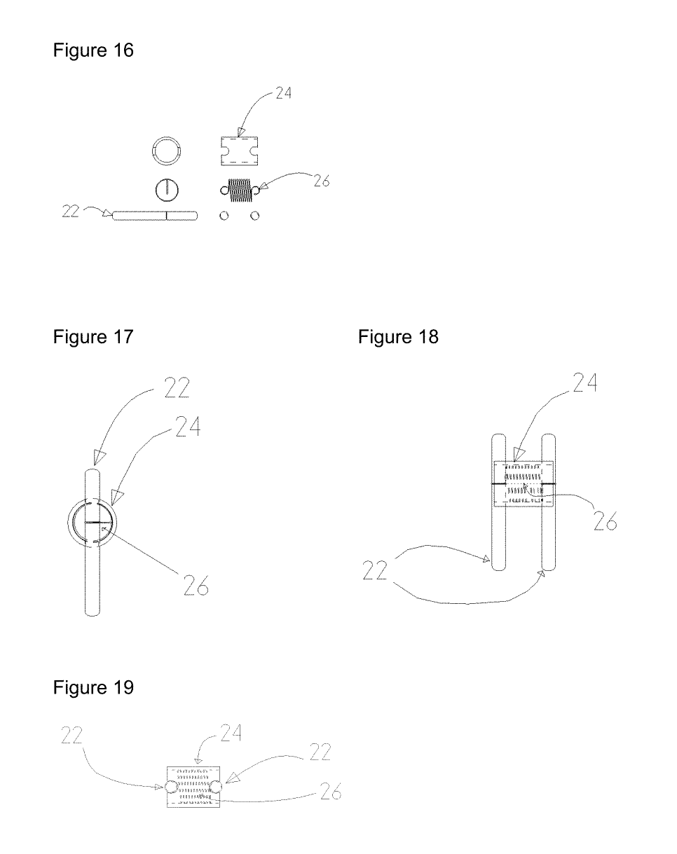

The arms of the device are attached to the ring by means of the knuckles and fingers shown in FIGS. 16, 17, 18 and 19. The fingers 22 are pulled together on either side of the ring by means of the knuckle finger tension spring 26 contained within the knuckle 24 which is in turn attached to the lower parts of the left arm 18 and right arm 20.

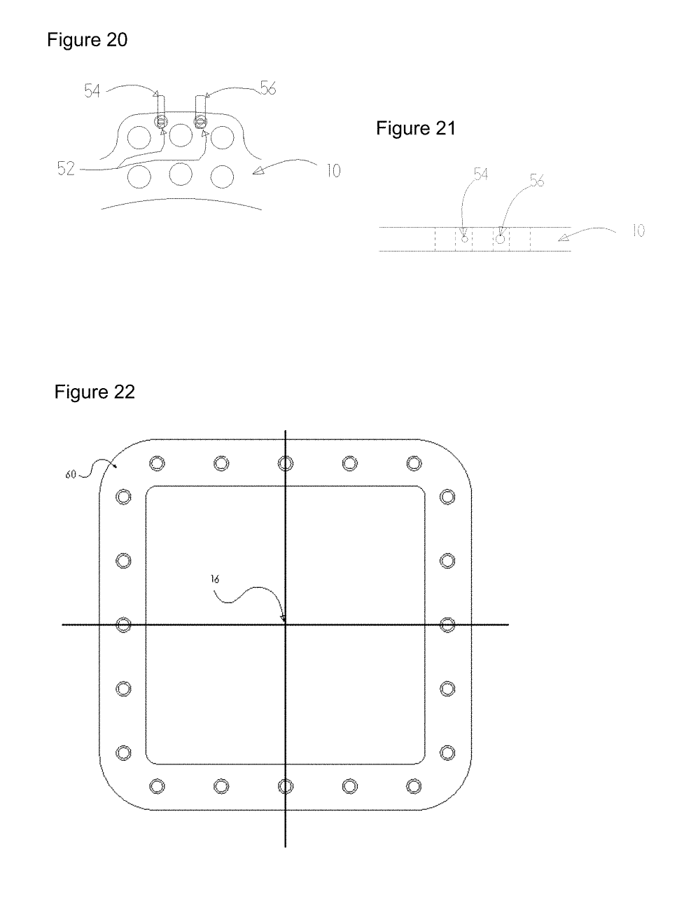

It is also possible to fit the laser module housing for the cross hair device 28 directly to the ring 10 i.e. without using the arms and platform provided that it has been appropriately prepared. FIGS. 20 and 21 show the reference ring 10 with the small hole 54 and large hole 56 with sprung ball grub screw 52 to accommodate the small registering pin 36 and large registering pin 34.

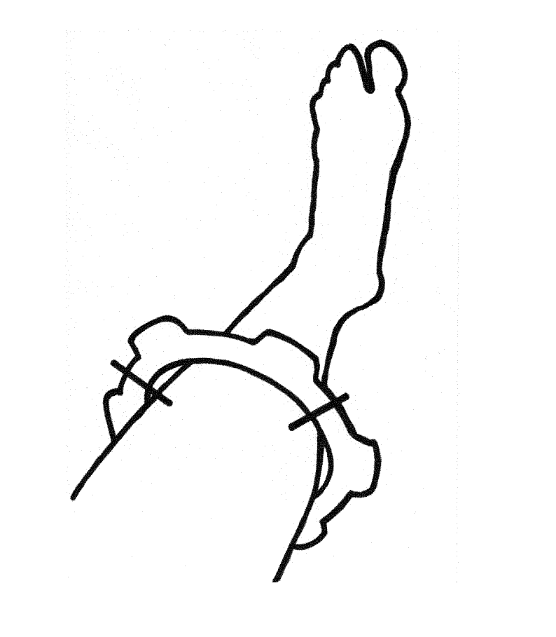

FIG. 22 shows the laser cross hair 16 as it appears when the correct alignment has been achieved, projected onto the cover of the x-ray machine 60.

It should be understood that some external fixator frames may have rings that are less than fully formed, for example the loop of the ring may not be complete. However for the purposes of this document a substantially formed, albeit less than complete, ring shape should nonetheless be considered to be a ‘ring’. Preferably the rings are circular although this is not essential.

While some preferred embodiments of the invention have been described by way of example it should be appreciated the modifications and improvements can occur without departing from the scope of the following claims.

1. A method of aligning an external fixator frame ring and a body part imaging device with respect to one another, comprising:
causing light to be projected from a position of the ring to indicate the planar disposition of the ring, and using the light to align the ring and imaging device with respect to one another.

2. A method according to claim 1, wherein a line of light is projected from the position of the ring and is used to align the ring and imaging device with respect to one another.

3. A method according to claim 1, wherein the body part imaging device comprises an x-ray imaging machine.

4. A method according to claim 1, wherein the light is emitted as a plane of light.

5. A method according to claim 1, wherein the light comprises laser light.

6. A method according to claim 1, wherein the light is in the same plane as the ring.

7. A method according to claim 1, wherein the light is projected by light emitting means secured to the ring.

8. A method according to claim 7, wherein the light emitting means projects two lines of light to create a cross-hair, the cross-hair indicating both the planar and rotational disposition of the ring.

9. An alignment device having:
attachment means; and
light emitting means;
the alignment device being secured to an external fixator frame ring by way of the attachment means and activated so that the emitting means projects a line of light towards a body imaging machine and the line is used to align the imaging machine with the fixator ring.

10. A device according to claim 9 wherein the body imaging device comprises an x-ray imaging machine.

11. A device according to claim 9, wherein the line of light is indicative of the planar disposition of the ring.

12. A device according to claim 9, wherein the light is emitted as a plane of light.

13. A device according to claim 9, wherein the line of light comprises laser light.

14. A device according to any one of claim 9, wherein the light emitting means also projects a second line of light towards the body imaging machine and wherein the second line is perpendicular to the first so that the two lines form a cross-hair.

15. A device according to any one of claim 9, wherein the attachment means comprises a pair of arms engaged with the ring.

16. A method according to claim 1, wherein the ring is substantially circular.

17. A device according to claim 9, wherein the ring is substantially circular.
